(19) United States
(12) Patent Application Publication
(10) Pub. No.: US 2026/0232928 A1
(43) Pub. Date: Aug. 13, 2026

(54) TWO-DOSE DOSAGE SYSTEM FOR A FLUID PRODUCT DISPENSING DEVICE

(71) Applicant: APTAR FRANCE SAS, Le Neubourg (FR)

(72) Inventor: Franck POULLAIN, La Haye Malherbe (FR)

(73) Assignee: APTAR FRANCE SAS, Le Neubourg (FR)

(21) Appl. No.: 19/155,817

(22) PCT Filed: Feb. 12, 2024
(86) PCT No.: PCT/FR2024/050187

§ 371 Date: Aug. 12, 2025

(30) Foreign Application Priority Data
Feb. 13, 2023 (FR) FR2301323

(51) Int. Cl. A61M 11/00; A61M 31/00

(52) U.S. Cl. CPC A61M 11/007; A61M 31/00; A61M 2202/0007; A61M 2202/04; A61M 2205/8275; A61M 2210/0618

(57) ABSTRACT

Two-dose dosage system for fluid product dispensing device, the device having a syringe-type reservoir containing fluid product, a piston secured to a piston rod being slidably mounted in the reservoir to dispense the fluid product. The two-dose dosage system including a fixing portion which is fixed on the piston rod, a dose-splitting mechanism to separate the actuation stroke of the piston rod into two half-strokes, each for dispensing a respective dose, and an energy accumulation mechanism to generate a resistance upon each actuation of said piston rod.

The present invention relates to a two-dose dosage system and a fluid product dispensing device comprising such a two-dose dosage system.

Fluid product dispensing devices of two-dose type are well known. They comprise a reservoir containing two doses of fluid product to be dispensed in two successive actuations. When the reservoir is of the syringe-type, with a piston secured to a piston rod which slides in a cylindrical reservoir, two-dose dosage systems exist which allow to separate the stroke of the piston into two half-strokes, one for each dose. These dosage systems are often complex and therefore, expensive to manufacture and to assemble, and generally require modification of the syringe and/or of the piston rod. In addition, they are generally not reusable.

Furthermore, when a nasal spray head is associated with a syringe-type reservoir, a disadvantage of the existing devices relates to the quality of the spray generated, which may depend on the actuation carried out by the user, particularly the speed and the actuation force, and may differ between the two doses. However, to guarantee a good reproducibility of the spray, it is desirable to generate a calibrated spray, i.e. the properties of which are substantially similar for the two doses, independently of the way in which the user actuates the device.

Documents U.S. Pat. Nos. 5,951,526, 9,878,104, 4,153,056 and US2013043282 describe prior-art devices.

The present invention aims to provide a two-dose dosage system and a fluid product dispensing device which does not reproduce the above-mentioned disadvantages.

The present invention aims particularly, to provide a two-dose dosage system which adapts to a fluid product dispensing device comprising a standard syringe-type reservoir, in particular, without having to modify the reservoir and/or the piston rod.

The present invention also aims to provide a two-dose dosage system which can be reused on several fluid product dispensing devices.

The present invention also aims to provide a two-dose dosage system and a fluid product dispensing device which allow to ensure a calibrated and reproducible spray for both doses, in particular, independently of the way in which the user actuates the device.

The present invention also aims to provide a two-dose dosage system and a fluid product dispensing device which guarantee a precise dosage for both doses.

The present invention also aims to provide a two-dose dosage system and a fluid product dispensing device which are simple and inexpensive to manufacture and to assemble.

The present invention therefore, aims to provides a two-dose dosage system for a fluid product dispensing device, said device comprising a syringe-type reservoir containing fluid product, a piston secured to a piston rod being slidably mounted in said reservoir to dispense said fluid product, said two-dose dosage system (20) comprising:
a fixing portion which fixes on said piston rod,
dose-splitting means to separate the actuation stroke of said piston rod into two half-strokes, each to dispense a respective dose, and
energy accumulation means, to generate a resistance during each actuation of said piston rod.

Advantageously, said fixing portion comprises two half-shells pivoting one on the other between an open position allowing assembly on said piston rod and a closed position in which said piston rod is enclosed between said two half-shells.

Advantageously, each half-shell comprises projecting fin.

Advantageously, said dose-splitting means comprise a pivoting assembly comprising a removable snap-fastening ring secured to a dosage abutment, said assembly pivoting about an axis between an active position and an inactive position.

Advantageously, in said active position, said snap-fastening ring is snap-fastened in a removable manner onto said piston rod or onto a portion fixed to said piston rod, said dosage abutment extending radially outwards from said piston rod such that, after dispensing a first dose, said dosage abutment abuts against said reservoir, preventing a continuation of the actuation stroke of said piston rod.

Advantageously, in said inactive position, said snap-fastening ring is released from said piston rod or from said portion fixed to said piston rod, and the assembly formed by said snap-fastening ring and said dosage abutment is pivoted about said axis away from said piston rod.

Advantageously, said energy accumulation means comprise a first pair of diametrically opposite axial tabs, each provided with a front end forming a first radial thickening, and a second pair of diametrically opposite axial tabs, each provided with a front end forming a second radial thickening, said second axial tabs being offset by 90° relative to said first axial tabs, said first axial tabs being longer than said second axial tabs, such that said first axial tabs generate an accumulation of energy for the first dose and said second axial tabs generate an accumulation of energy for the second dose.

Advantageously, said first axial tabs are arranged in a first pair of diametrically opposite longitudinal grooves of said piston rod such that only said front ends project radially out of said grooves, and said second axial tabs are arranged in a second pair of diametrically opposite longitudinal grooves of said piston rod, such that only said front ends project radially out of said grooves.

Advantageously, said two-dose dosage system is moulded of a single one-piece.

The present invention also aims, to provide a fluid product dispensing device comprising a syringe-type reservoir, a piston slidably mounted in said reservoir, said piston being secured to a piston rod, said device comprising a two-dose dosage system as described above, said dosage system being assembled on said piston rod.

Advantageously, the device comprises a nasal spraying head provided with a spraying orifice mounted on said reservoir.

These advantages and characteristics and others of the present invention will appear more clearly during the detailed description below, made in reference to the accompanying drawings, given as non-limiting examples, and in which:

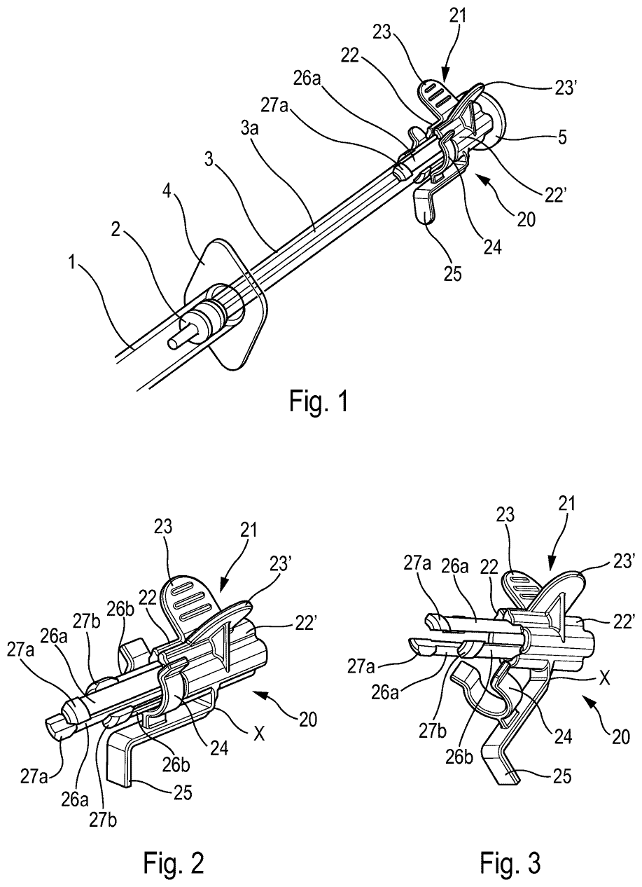

FIG. 1 is a partial schematic perspective view of a fluid product dispensing device comprising a two-dose dosage system according to an advantageous embodiment,

FIG. 2 is a detailed perspective view, showing the two-dose dosage system in a first position,

FIG. 3 is a view similar to that of FIG. 2, showing the two-dose dosage system in a second position,

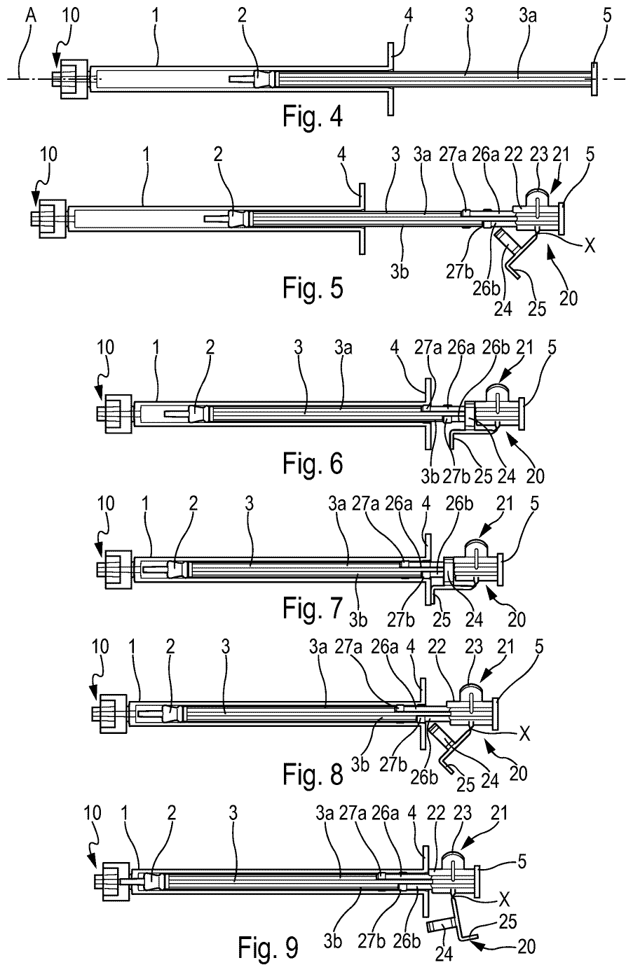

FIG. 4 is a schematic side view of a fluid product dispensing device,

FIG. 5 is a view similar to the that of FIG. 4, after assembly of the two-dose dosage system on the piston rod,

FIG. 6 is a view similar to that of FIG. 5, after dispensing the first dose,

FIG. 7 is a view similar to that of FIG. 6, after dispensing the first dose,

FIG. 8 is a view similar to that of FIG. 7, before dispensing the second dose, and

FIG. 9 is a view similar to that of FIG. 8, after dispensing the second dose.

The terms “axial” and “radial” refer to the longitudinal central axis A of the dispensing device, shown in FIG. 4. The terms “front” and “rear” refer to the direction of flow of the fluid product during the actuation of the device.

The present invention will be described below with reference to an embodiment regarding to a fluid product dispensing device provided with a spray head, but it is understood that other embodiments could be considered, particularly, conventional injection syringes, comprising a needle for dispensing the fluid product.

FIG. 4 very schematically represents a spray head 10 assembled on a syringe-type reservoir 1. A removable protective cap may be assembled on the spray head before using the device. The reservoir 1 contains two doses of fluid product, generally a liquid, which are dispensed in two successive actuations.

The reservoir 1 is preferably formed by a hollow cylinder provided with a collar 4 at its rear axial end edge which surrounds a rear opening of the reservoir.

Conventionally, suitable dispensing means are provided in the reservoir 1 to dispense its contents. Typically, these dispensing means comprise a piston 2 slidably mounted in the reservoir 1 and secured to a piston rod 3 actuated by the user at the time of the actuation.

The piston rod 3 comprises, at its rear axial end edge, a bearing flange 5 on which the user presses during the actuation. The piston rod 3 comprises a cross-shaped cross section, with a first pair of diametrically opposite longitudinal grooves 3a and a second pair of diametrically opposite longitudinal grooves 3 b offset by 90° relative to the first pair of longitudinal grooves 3a. The function of these grooves 3a, 3b will be explained below.

The spray head 10 can conventionally comprise a body 11 provided with a spraying orifice. Preferably, the spray head 10 is of the nasal type, with an extended body to penetrate into a nostril and the spraying orifice arranged at the front axial end of said body, in the flow direction of the fluid product during the actuation. Advantageously, an insert can be arranged upstream of the spraying orifice to generate a spray, for example by means of a well-known spraying profile. Other types of spray head are also considered.

According to the invention, a two-dose dosage system 20 is provided which is fixed on the piston rod 3, outside the reservoir 1.

The two-dose dosage system 20 comprises a fixing portion 21 which is fixed on the piston rod 3. Preferably, this fixing is removable to allow the reuse of the dosage system 20.

In the example shown, which is not limiting, this fixing portion 21 comprises two half-shells 22, 22′ pivoting on one another between an open position allowing assembly on the piston rod 3 and a closed position in which the piston rod 3 is enclosed between the two half-shells. Each half-shell 22, 22′ comprises a projecting fin 23, 23′ to facilitate the manipulation of the fixing portion 21 and its assembly on the piston rod 3.

The two-dose dosage system 20 comprises dose-splitting means 24, 25 to separate the actuation stroke of the piston rod 3 into two half-strokes, each for dispensing a respective dose.

In the example shown, which is not limiting, these dose-splitting means comprise a pivoting assembly comprising a removable snap-fastening ring 24 secured to a dosage abutment 25. This assembly is pivoting about an axis X between an active position, shown in FIGS. 1, 2, 6, and 7, and an inactive position, shown in FIGS. 3, 5, 8, and 9.

In the active position, the snap-fastening ring 24 is snap-fastened in a removable manner on the piston rod 3 or on a portion of the two-dose dosage system 20 which is fixed to the piston rod 3. In this active position, the dosage abutment 25 extends radially outwards from the piston rod 3, such that after dispensing a first dose, it abuts against the collar 4 of the reservoir 1, as can be seen in FIG. 7, preventing the continuation of the actuation stroke of the piston rod 3.

In the inactive position, the snap-fastening ring 24 is released from the piston rod 3 or the portion of the two-dose dosage system 20 which is fixed to the piston rod 3, and the assembly formed from the snap-fastening ring 24 and the dosage abutment 25 is pivoted about the axis X away from the piston rod 3. In this inactive position, the dosage abutment 25 no longer abuts against the collar 4 of the reservoir 1, as can be seen in FIGS. 8 and 9, and the actuation rod 3 can be actuated over a second stroke portion to dispense the second dose.

The two-dose dosage system 20 comprises energy accumulation means 26a, 27a, 26b, 27b, to generate a resistance to the actuation of the piston rod 3. The user must therefore, apply to the piston rod 3 a force at least equal to a predetermined threshold, which generates an accumulation of energy in his hand that will be released abruptly when this resistance is overcome. This allows to guarantee, at each actuation, a dispensing of the complete dose with reproducible dispensing parameters.

In the example shown, which is not limiting, these energy accumulation means comprise a first pair of diametrically opposite axial tabs 26a, each provided with a front end forming a first radial thickening 27a. These first axial tabs 26a are arranged in the first pair of diametrically opposite longitudinal grooves 3a of the piston rod 3 in such a way that only the front ends 27a project radially out of said grooves 3a, as can be seen particularly in FIG. 5. Correspondingly, the energy accumulation means comprise a second pair of diametrically opposite axial tabs 26b, each provided with a front end forming a second radial thickening 27b. These second axial tabs 26b are offset by 90° relative to the first axial tabs 26a. Thus, these second axial tabs 26b are arranged in the second pair of diametrically opposite longitudinal grooves 3b of the piston rod 3, such that only the front ends 27b project radially out of said grooves 3b. Clearly, the first axial tabs 26a are longer than the second axial tabs 26b, such that the first axial tabs 26a generate an accumulation of energy for the first dose and the second axial tabs 26b generate an accumulation of energy for the second dose.

Alternatively, the energy accumulation means could comprise only one first axial tab and only one second axial tab. Other embodiments are also possible.

Advantageously, the two-dose dosage system 20 is moulded of a single one-piece.

The operation of the invention will be described below with reference to FIGS. 4 to 9 which illustrate an actuation sequence.

In FIG. 4, the reservoir 1 of the dispensing device has been filled with fluid product.

The user then assembles the two-dose dosage system 20 on the piston rod 3, preferably in abutment with the bearing flange 5 of the piston rod 3, as shown in FIG. 5.

To calibrate the dosage to be dispensed during two successive actuations, the piston rod 3 is moved axially relative to the reservoir 1 to bring the front end 27a of the first axial tabs 26a into abutment against the rear opening of the reservoir, at the collar 4. It should be noted that FIGS. 4 and 5 are not necessarily to scale, and the axial movement of the piston rod 3 to bring the first axial tabs 26a into abutment against the reservoir, may be very short.

FIG. 6 shows the device in position before dispensing the first dose. In this position, the dose-splitting means are in the active position.

To dispense the first dose, the user presses.

The end of the first actuating stroke is formed by the dosage abutment 25 which cooperates with the collar 4 of the reservoir 1, as can be seen in FIG. 7.

Simultaneously, the front end 27b of the second axial tabs 26b comes into abutment against the rear opening of the reservoir, at the collar 4.

To be able to dispense the second dose, the user must then move the dose-splitting means to the inactive position, as can be seen in FIG. 8.

The user then presses again on the bearing flange 5 of the piston rod 3 so as to move it in the reservoir 1. When this bearing force reaches the predetermined threshold, the second thickenings 27b of the second axial tabs 26b deform to penetrate into the reservoir 1, allowing an axial movement of the piston rod 3 and therefore, of the piston 2, generating the dispensing of the second dose.

The end of the second actuation stroke is formed by the half-shells 22, 22′ which come to abut on the collar 4 of the reservoir 1, as can be seen in FIG. 9.

If desired, the two-dose dosage system 20 can be removed from the device of FIG. 9, and assembled on another fluid product dispensing device.

Although the present invention has been described above with reference to several embodiments thereof, it is understood that the invention is not limited to the embodiments described and represented in the drawings, but, on the contrary, any useful modification could be applied thereto by a person skilled in the art, without going beyond the scope of the present invention, such as defined by the accompanying claims.

1. A two-dose dosage system for fluid product dispensing device, said device comprising a syringe-type reservoir containing fluid product, a piston secured to a piston rod being slidably mounted in said reservoir to dispense said fluid product, characterised in that said two-dose dosage system comprises:
a fixing portion which is fixed on said piston rod,
dose-splitting means to separate the actuation stroke of said piston rod into two half-strokes, each for dispensing a respective dose, and
energy accumulation means, to generate a resistance upon each actuation of said piston rod.

2. The system according to claim 1, wherein said fixing portion comprises two half-shells pivoting one on another between an open position allowing assembly on said piston rod and a closed position in which said piston rod is enclosed between said two half-shells.

3. The system according to claim 2, wherein each half-shell comprises a projecting fin.

4. The system according to claim 1, wherein said dose-splitting means comprise a pivoting assembly comprising a removable snap-fastening ring secured to a dosage abutment, said assembly pivoting about an axis between an active position and an inactive position.

5. The system according to claim 4, wherein, in said active position, said snap-fastening ring is snap-fastened in a removable manner on said piston rod or onto a portion fixed to said piston rod, said dosage abutment extending radially outwards from said piston rod, such that after dispensing a first dose, said dosage abutment abuts against said reservoir, preventing a continuation of the actuation stroke of said piston rod.

6. The system according to claim 5, wherein, in said inactive position, said snap-fastening ring is released from said piston rod or from said portion fixed on said piston rod, and the assembly formed by said snap-fastening ring and said dosage abutment is pivoted about said axis away from said piston rod.

7. The system according to claim 1, wherein said energy accumulation means comprise a first pair of diametrically opposite axial tabs, each provided with a front end forming a first radial thickening, and a second pair of diametrically opposite axial tabs, each provided with a front end forming a second radial thickening, said second axial tabs being offset by 90° relative to said first axial tabs, said first axial tabs being longer than said second axial tabs, such that said first axial tabs generate an accumulation of energy for the first dose and said second axial tabs generate an accumulation of energy for the second dose.

8. The system according to claim 7, wherein said first axial tabs are arranged in a first pair of diametrically opposite longitudinal grooves of said piston rod such that only said front ends project radially out of said grooves, and said second axial tabs are arranged in a second pair of diametrically opposite longitudinal grooves of said piston rod such that only said front ends project radially out of said grooves.

9. The system according to claim 1, wherein said two-dose dosage system is moulded of a single one-piece.

10. A fluid product dispensing device comprising a syringe-type reservoir, a piston slidably mounted in said reservoir, said piston being secured to a piston rod, characterised in that said device comprises a two-dose dosage system according to claim 1, said dosage system being assembled on said piston rod.

11. The device according to claim 10, comprising a nasal spray head provided with a spray orifice mounted on said reservoir.
